(19) United States
(12) Patent Application Publication
(10) Pub. No.: US 2011/0021997 A1
(43) Pub. Date: Jan. 27, 2011

(54) Catheter securement device

(76) Inventors: Kurt Kyvik, Ocala, FL (US); Scott Ryan, Ocala, FL (US)

Correspondence Address: ROGERS TOWERS, P.A., 1301 RIVERPLACE BOULEVARD, SUITE 1500, JACKSONVILLE, FL, 32207, US

(21) Appl. No.: 12/804,422

(22) Filed: Jul. 21, 2010

Related U.S. Application Data
(60) Provisional application No. 61/271,430, filed on Jul. 21, 2009.

(51) Int. Cl. A61M 25/02

(52) U.S. Cl. 604/180

(57) ABSTRACT

An improved catheter securement device, the device having a main body assembly for securing a catheter housing, the assembly adapted to adhesively adher to the skin of a patient, wherein the improvement is a thin, flexible base extension member that is adapted to adher to the patient's skin, the base extension member being provided with an elongated slit dividing the base extension member into two leg portions. The catheter securement device is adhered to the patient such that the venous catheter portion extends through the slit at the insertion site when the catheter housing is properly retained. Preferably, the adhesive utilized to adhere the base extension member to the patient's skin incorporates an anti-microbial composition to combat microorganisms in the area of the insertion site.

This application claims the benefit of U.S. Provisional Patent Application Ser. No. 61/271,430, filed Jul. 21, 2010.

### BACKGROUND OF THE INVENTION

This invention relates generally to the field of medical devices referred to as catheter securement devices, a catheter being defined herein to include a small diameter tube that is inserted through a person's skin and into a vein to deliver or remove fluids such as blood, saline solutions, medications, etc. A catheter securement device typically comprises a flexible sheet member adhesively adhered to the patient's skin, the device having means to retain the catheter housing, often a coupling or junction member for connecting one tube to another or to multiple tubes, in a relatively stable manner such that it is less likely for the catheter to be accidentally pulled from the patient.

Catheter securement devices are usually located a short distance from the insertion site, such that the securement device has a minimal or reduced effect in securing the catheter tube in place at the actual insertion site. Furthermore, the catheter insertion site is susceptible to infection since the skin has been punctured. It is an object of this invention to provide an improved catheter securement device that addresses both problems by reducing the likelihood of accidental dislodgement of the catheter and which provides antimicrobial protection.

### SUMMARY OF THE INVENTION

The invention is an improved catheter securement device, the device comprising a main body for securing the catheter housing, the main body being adhesively adhered to the skin of a patient, wherein the improvement comprises a base extension member comprising a thin, flexible base member that is also adhered to the patient's skin, the base extension member being provided with an elongated slit dividing the base extension member into two leg portions. The catheter securement device is adhered to the patient such that the venous catheter assembly extends through the slit and between the two leg portions of the base extension member at the insertion site. The leg portions may be overlapped to better secure the catheter assembly at the insertion site and to completely encircle the catheter assembly. Preferably, the adhesive utilized to adhere the base extension member to the patient's skin contains an anti-microbial composition to combat microorganisms in the area of the insertion site.

### BRIEF DESCRIPTION OF THE DRAWINGS

FIG. 1 is an end view of the main base portion of an embodiment of the device, shown prior to adhesion to the patient.

FIG. 2 is an end view of the main portion of FIG. 1 with the release liners having been removed, a catheter housing being retained by the device in known manner and the device being adhered to the patient's skin.

FIG. 3 is a partial side view of the base extension member, shown prior to adhesion to the patient.

FIG. 4 is a top view of an embodiment of the device.

FIG. 5 is a view showing the showing an embodiment of the device as adhered to the skin of a patient.

### DETAILED DESCRIPTION OF THE INVENTION

With reference to the drawings, the invention will now be described in detail with regard for the best mode and the preferred embodiment.

In general, the catheter securement device comprises a base member 10 and a base extension member 21. As shown in the embodiment illustrated herein, the base extension member 21 extends generally perpendicularly to the base member 10, such that the device has a general T-shaped configuration. This configuration is especially suitable for application of the device to the arm of a patient, as shown in FIG. 5. Variations in the overall shape and configuration of the device are possible without departing from the nature of the invention.

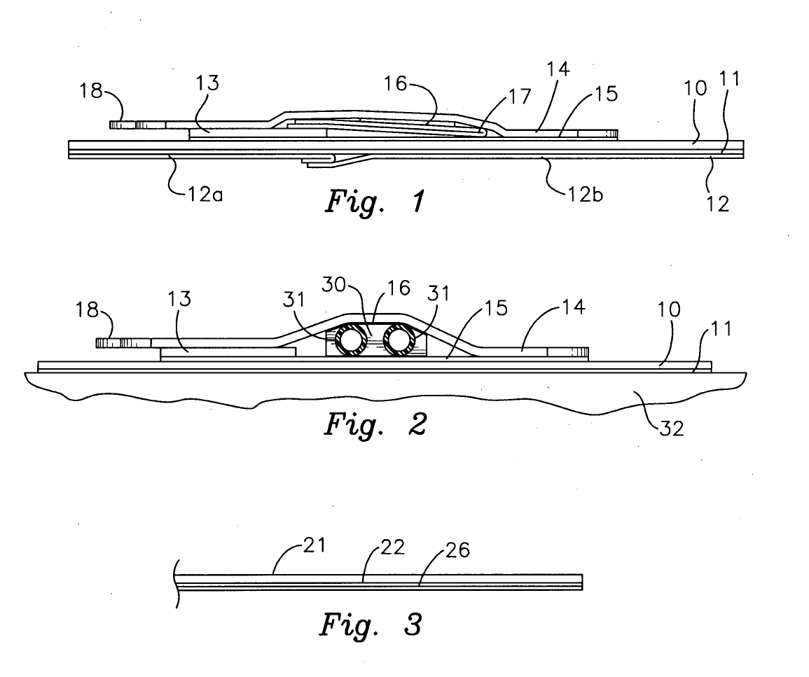

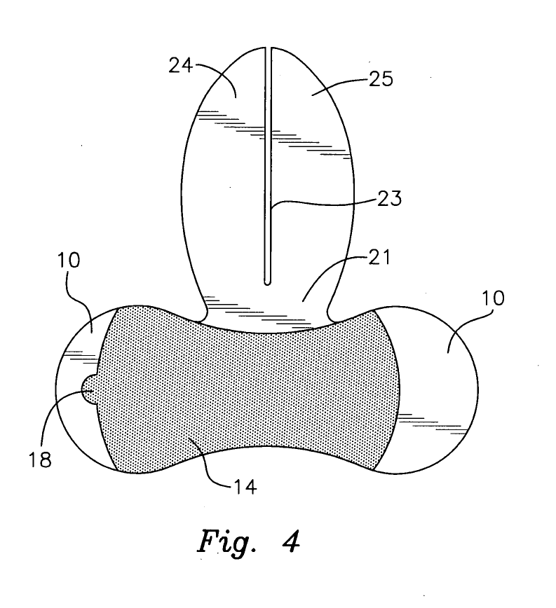

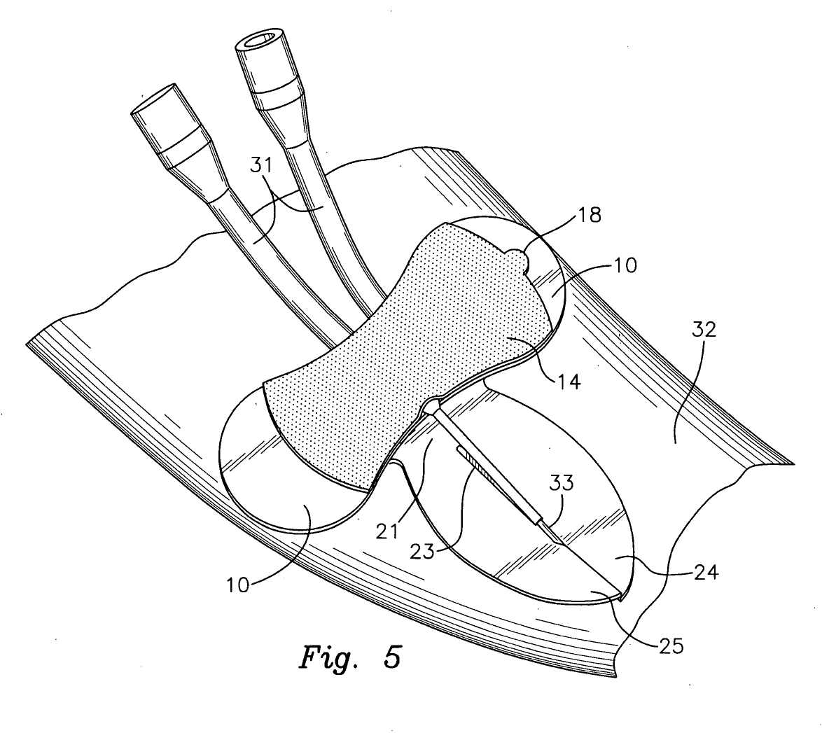

The base member 10 is part of an assembly that directly retains and secures the catheter housing 30, which in the embodiment shown in FIG. 2 for example comprises a connector, splitter or manifold that allows two tubes 31 to be joined to a single tube extending from the catheter assembly 33. The venous catheter assembly 33 is represented herein as a combination of a rigid tubular puncturing member, a junction means and a flexible tube, but it is to be understood that the invention is suitable for use with a large number of known catheter housings and catheter assemblies. The particular mechanism or method for securement of the catheter housing 30 by the base member 10 assembly is not crucial to the invention, and various structures and methods for accomplishing this purpose are known in the art.

In the embodiment shown in the drawings, the mechanism and method of securing the catheter housing 30 is an assembly comprising a thin, flexible, generally elongated, base member 10, preferably transparent or semi-transparent, having an adhesive layer 11 applied to its bottom side for adherence of the base member 10 to the patient's skin 32. A lower retention adhesive layer 15 is applied to the top of the base member 10. A hook sheet member 13 containing the hooks of a hook-and-loop fastener system is adhered by the lower retention adhesive layer 15 onto the base member 10, with the hook sheet member 13 being positioned off-center. An elongated, flexible, loop sheet member 14 containing the loops of a hook-and-loop fastener system has one end adhered to the lower retention adhesive layer 15 and extends across the center of the base member 10 so as to overlap the hook sheet member 13. A pull tab member 18 may be provided on this free end of the loop sheet member 14. In this manner the free end of the loop sheet member 14 may be released from and re-attached to the hook sheet member 15, thereby defining a strap that is used to overlay and secure the catheter housing 30, as shown in FIG. 2. Preferably, an upper retention adhesive layer 16 is provided on the underside of the central portion of the loop sheet member 14, such that the catheter tubes 31 and catheter housing 30 are further retained by adhesion with the upper retention adhesive layer 16 and the lower retention adhesive layer 15. An additional adhesive layer may be applied atop the central portion of the lower retention adhesive layer 15 if desired. As shown in FIG. 1, a base release liner 12 is provided on the exposed surface of the base adhesive layer 11, which may be divided into a first portion 12a and a second portion 12b for easier removal of the base release liner 12 prior to adherence to the patient's skin. Likewise, an interior release liner 17 may be provided on the exposed central region of the lower retention adhesive layer 15 prior to insertion of the catheter tubes 31.

The improvement to catheter securement devices of the type similar to the embodiment described above comprises the addition of a thin, flexible, generally elongated, base extension member 21, preferably transparent or semi-transparent, that extends from the base member 10. In the embodiment shown in the drawings, the base extension member 21 will extend generally perpendicularly to the longitudinal axis of the base member 10 assembly. In this manner the base extension member 21 extends in the direction substantially parallel to the direction of the catheter tubes 31 extending from the venous catheter assembly 33 at the insertion site. The base extension member 21 is provided with an adhesive layer 22 on its bottom to adhere the base extension member 21 to the patient's skin in similar manner to the base adhesive layer 11. A separate release layer 26 may be provided on the exposed underside of the extension adhesive layer 22 for removal prior to application to the patient, or this extension release layer 26 may be formed as a component of the base release layer 12.

The base extension member 21 is provided with an elongated slit or slot 23 extending to its free end. The slit 23 divides the base extension member to define a first leg member 24 and a second leg member 25. The slit 23 allows the catheter securement device to be positioned, with the catheter housing 30 being retained, such that the first and second leg members 24 and 25 extend beyond the catheter insertion site on opposing sides, the venous catheter assembly 33 extending through the slit 23. Preferably, the portions of the extension leg members 24 and 25 extending beyond the catheter insertion site are slightly twisted toward each other on application to the patient's skin 32 such that the free ends of the extension leg members 24 and 25 overlap. This overlap serves creates a 360 degree enclosure to better secure the catheter assembly 33 to the patient's skin 32, both from physical restraint of the material comprising the extension member 21 as well as by contact between the catheter assembly 33 and a portion of the adhesive layer 22 that is wrapped across the catheter assembly 33.

In the most preferred embodiment the extension adhesive layer 22 is provided with an anti-microbial agent to ward off infection at the catheter insertion site, either applied as a coating to the exposed side of the adhesive layer 22 or incorporated into the adhesive layer 22 itself. While many known anti-microbial agents, such as for example chlorohexidine gluconate, may be suitable for this purpose, preferable agents to be incorporated in or applied to the extension adhesive layer 22 include silver ions or Manuka honey (e.g., MEDIHONEY brand by Derma Sciences, Inc.), as these agents offer less toxic efficaciousness in comparison to other anti-microbial agents. With the extension leg members 24 and 25 overlapped, the anti-microbial zone of protection extends for 360 degrees about the catheter insertion site. The anti-microbial agent may be localized in the region near to the slit 23 or may be incorporated across the entire extension adhesive layer 22, and even across the base adhesive layer 11 as well.

It is to be understood that equivalents and substitutions to elements and structures set forth above, which are not intended to be limiting unless necessary for patentability, may be obvious to those of ordinary skill in the art, and therefore the true scope and definition of the invention is to be as set forth in the following claims.

We claim:

1. A catheter securement device comprising:
a base member comprising a base adhesive layer adapted for attachment of said base member to a person's skin, said base member adapted to secure a catheter housing connected to a catheter assembly inserted into a person's skin at an insertion site;
a base extension member joined to said base member in a manner whereby with said base member securing the catheter housing, said base extension member extends beyond the insertion site of said catheter assembly;
said base extension member comprising a slit, said slit dividing said base extension member into a first leg portion and a second leg portion, wherein said slit is adapted to receive the catheter assembly between said first and second leg portions;
said base extension member further comprising a base extension adhesive layer adapted for attachment of said base extension member to a person's skin.

2. The device of claim 1, further comprising an anti-microbial agent.

3. The device of claim 2, wherein said anti-microbial agent is incorporated within said base extension adhesive layer.

4. The device of claim 2, wherein said anti-microbial agent is a coating on said base extension adhesive layer.

5. The device of claim 1, wherein said base extension member is thin, flexible and elongated.

6. The device of claim 5, wherein said first and second leg portions are overlapping when said catheter assembly is positioned between said first and second leg portions.

7. The device of claim 2, wherein said base extension member is thin, flexible and elongated.

8. The device of claim 7, wherein said first and second leg portions are overlapping when said catheter assembly is positioned between said first and second leg portions.

9. The device of claim 2, wherein said anti-microbial agent is incorporated within said base extension adhesive layer and said base adhesion layer.

10. The device of claim 2, wherein said anti-microbial agent is a coating on said base extension adhesive layer.

11. A catheter securement device adapted to secure a catheter housing connected to a catheter assembly inserted into a person's skin, said device comprising:
an assembly comprising a base member and a base adhesive layer;
a base extension member joined to said base member, said base extension member comprising a base extension adhesive layer and a slit, said slit dividing said base extension member into a first leg portion and a second leg portion;
wherein said base extension member is composed of a thin, flexible material whereby said first and said second leg portions are adapted to overlap to surround the catheter assembly.

12. The device of claim 11, further comprising an anti-microbial agent.

13. The device of claim 12, wherein said anti-microbial agent is incorporated within said base extension adhesive layer.

14. The device of claim 12, wherein said anti-microbial agent is a coating on said base extension adhesive layer.

15. A catheter securement device in combination with a catheter housing connected to a catheter assembly inserted into a person's skin, said device comprising:
an assembly comprising a base member and a base adhesive layer, said assembly receiving and securing said catheter housing;
a base extension member joined to said assembly, said base extension member composed of a thin, flexible material, said base member comprising a base extension adhesive layer and a slit, said slit dividing said base extension member into a first leg portion and a second leg portion, said slit receiving said catheter housing between said first and second leg portions with said first and second leg portions overlapped.

16. The device of claim 15, further comprising an anti-microbial agent.

17. The device of claim 16, wherein said anti-microbial agent is incorporated within said base extension adhesive layer.

18. The device of claim 16, wherein said anti-microbial agent is a coating on said base extension adhesive layer.

19. The device of claim 16, wherein said anti-microbial agent is incorporated within said base extension adhesive layer.

20. The device of claim 16, wherein said anti-microbial agent is a coating on said base extension adhesive layer.
